(19) United States
(12) Patent Application Publication
(10) Pub. No.: US 2011/0066179 A1
(43) Pub. Date: Mar. 17, 2011

(54) MOBILE SUBSTRATE ATTACHMENT DEVICE

(75) Inventors: Bryan Greener, Heslington (GB); John Hicks, Heslington (GB)

(73) Assignee: Smith & Nephew PLC, Heslington, York, EN (GB)

(21) Appl. No.: 12/918,072

(22) PCT Filed: Feb. 19, 2009
(86) PCT No.: PCT/GB2009/000452

§ 371 Date: Nov. 22, 2010

(30) Foreign Application Priority Data
Feb. 20, 2008 (GB) 0803059.5

(51) Int. Cl. A61B 17/00

(52) U.S. Cl. 606/213

(57) ABSTRACT

The present invention provides an attachment device′ having a substrate facing surface, wherein the substrate facing surface is provided with at least one flexible protrusion extending therefrom, said protrusion forming a flexible bridge between the substrate facing surface and a flexible substrate.

This application claims priority to UK patent application No. 0803059.5 filed on 20 Feb. 2008 entitled “Mobile Substrate Attachment Device”, the entire contents of which are hereby incorporated by reference.

### FIELD OF THE INVENTION

The invention relates generally to extensible, flexible devices for attachment to mobile surfaces, thereby enabling, for example, the attachment of non-extensible, inflexible devices to mobile surfaces.

### BACKGROUND TO INVENTION

Attachment of one object to another via a contact surface is a paradigm for the assembly of multiple component systems. The adjoining components can be naturally or synthetically derived and may be alive, dead or inanimate matter. Common joining methods belong to one of two large families: adhesive and mechanical. Adhesives include chemical reaction adhesives (e.g. cyanoacrylates, anaerobics, acrylics, epoxies, polyurethanes, polyimides, phenolics and silicones) and physical reaction (e.g. hot-melts, plastisols, rubbers, PVAs and pressure sensitives) adhesives. Mechanical methods include interpenetrating, interlocking and interference mechanisms (e.g. riveting, bolting, screwing, nailing, jointing, zipping, stitching, buttoning, hook- and loop-fastening and suction). It can be generalised from these examples that adhesive methods and mechanical methods are commonly applied to join physically inflexible objects together. Meanwhile, for highly flexible materials like fabrics, mechanical methods dominate.

Against this background, it is perhaps not surprising that mechanical methods of attachment dominate medical practices concerning soft tissues (suturing, stapling). Even for attachment to hard tissue, a strong mechanical element is maintained (interference fit of screws and nails into bone, dental and bone cements). The use of medical adhesives for the joining of tissue is very limited in comparison to mechanical methods. An exception to this is in the application of devices to the human skin: such devices, including drug delivery patches, wound dressings and ostomy pouches, are conventionally attached using adhesives. This is because these devices rarely remain in place for extended periods and also because currently available mechanical attachment methods are considered too painful (e.g. staples) for non-surgical applications.

The mechanical joining of soft tissue by current methods is not ideal because the materials applied (e.g. nylon sutures, metal staples) are not mechanically well matched to the tissue(s) being joined. The mechanical mismatch creates anisotropic and unnatural forces within the tissues and can cause local tissue death and further complications (e.g. site for bacterial colonisation). Similarly, the adhesive joining of soft tissues or the adhesive attachment of devices to the skin by current methods is not ideal because of mechanical mismatching. For example, topical devices on the skin frequently become dislodged before the end of use.

In conclusion, current paradigms for the joining of soft tissues or attachment to soft tissues are mechanically mismatched to the tissues to which they are applied; this leads to abnormal mechanical loading and either device failure, tissue damage or both. The purpose of this invention is the re-design of the interface of attachment to soft tissue. The starting point for re-examination is an understanding of the mechanics of soft tissue itself. Key to our insight is the fact that soft tissue is unusual because, in contrast to most man-made materials, it is flexible, not very stretchy but highly mobile. These properties are the result of the geometry and multilaminate construction of our soft tissues, particularly the skin. It is common misconception that skin is stretchy; skin is not very stretchy, as new mothers and crash dieters can attest to. Our skin is however mobile because we carry an excess area of it to accommodate our body's extensive range of movement.

Woven, non-woven and knitted materials provide good examples of man-made constructs that can exhibit similar properties to soft tissue, hence their extensive use in clothing. Our clothing, able to slide freely on the surface of our skin, forms a loose bilaminate on our surface, localised by topological constraint; this is not unlike our skin, which can itself become separated from the body due to high shear trauma. The attachment of small devices to the skin does not require attachment to a bespoke undergarment; circumferential bands can meet some of these needs but attachment of devices to the torso requires an alternative strategy. The need to be able to position a portable device to any location of the human body necessitated the use of pressure sensitive adhesives, of the sort used on Band-Aid™ or Elastoplast™. The application of such adhesives to medical devices for topical attachment can still lead to complications of mechanical mismatching, for example shear-force blistering.

The present invention provides an extensible, flexible device for attachment to mobile surfaces; including the attachment of non-extensible, inflexible devices to mobile surfaces.

### SUMMARY OF INVENTION

The principle of the invention is to construct a device having a surface that it is capable of the same or greater extension and flexibility than the substrate to which it is attached. This is achieved by utilising a device having a surface with discontinuous contact points. The contact points, which contact only a small percentage of the substrate, are connected by a device surface path length in excess of that which can be generated between the same points on the flexible substrate.

Thus according to a first aspect of the invention there is provided an attachment device having a substrate facing surface, wherein the substrate facing surface is provided with at least one flexible protrusion extending therefrom, said protrusion forming a flexible bridge between the substrate facing surface and a flexible substrate.

This aspect of this invention concerns a device for attachment to a flexible substrate. Here the word ‘flexible’ is used to describe mobile, extensible, flexible substrates.

The device may be used to join two or more spatially separated surfaces, at least one of which is a flexible.

The device may be used to join two or more spatially separated surfaces, at least two of which are flexible. As such in an embodiment of the invention the attachment device is provided with a first substrate facing surface and a second substrate facing surface, wherein both substrate facing surfaces are provided with at least one flexible protrusion extending therefrom, said protrusion forming a flexible bridge between the first substrate facing surface and a first flexible substrate and between the second substrate facing surface and a second flexible substrate.

In embodiments of the invention the flexible surfaces may be internal or external surfaces of the human or animal body. For example, surfaces of soft tissues. The term soft tissue refers to tissues that connect, support, or surround other structures and organs of the body. Soft tissue includes muscles, tendons, ligaments, fascia, nerves, fibrous tissues, fat, blood vessels and synovial tissues.

The device may be constructed of any suitable flexible material; for example an articulated rigid section material or an inherently flexible material. For ease of construction, the device is preferably fabricated from a mouldable material. Preferably, the mouldable material is elastic when set in its final geometry. The material may be, for example, a thermoplastic, heat-curable or photo-curable polymer. Thermoplastic or heat curable polyurethanes and silicone-based polymers are examples of suitable polymers.

Preferably the material is biocompatible.

The protrusion can be integral with the substrate facing surface, for example moulded as part of the device. Alternatively the protrusion can be attached to the substrate facing surface, for example by a′ suitable adhesive. In this embodiment the protrusion can made of the same or a different material to the remainder of the device.

In embodiments of the invention the substrate facing surface is provided with a plurality of protrusions.

The protrusion(s) can extend substantially perpendicularly from the substrate facing surface. Alternatively the protrusions can extend at an angle from the substrate facing surface.

The protrusion(s) are of suitable dimension, geometry and flexibility to ensure that the contact point spacing can be extended without resulting in detachment from the flexible substrate. This requires that the joining force generated between the device and the flexible substrate is not overcome by the mechanical effect of extension or compression.

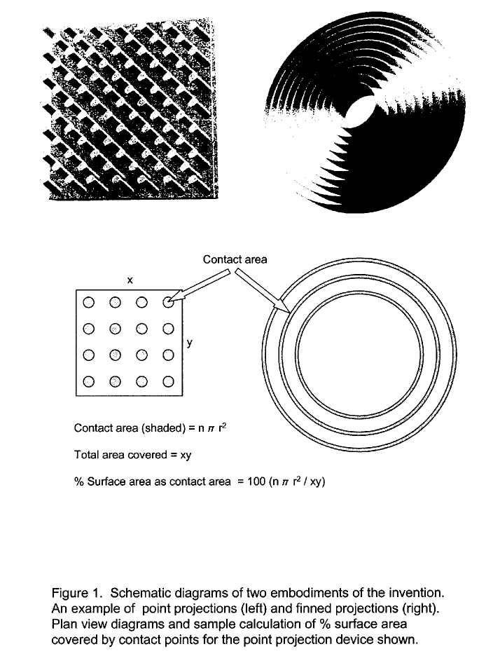

Examples of suitable geometries for the protrusion(s) include cylindrical or concentric rings of a tapered, thin-walled element as illustrated in FIG. 1. It is also envisaged that the protrusion can be in the form of a single coil-shaped protrusion extending from the centre of the device.

The protrusion(s) are sufficiently flexible such that extension of protrusion-to-protrusion spacing can be achieved with a mechanical force that does not result in the displacement of the device from the substrate.

In embodiments of the invention the geometry of the plurality of protrusions is identical.

Each protrusion has a substrate contacting surface. In embodiments of the invention this surface is substantially parallel to the substrate facing surface. Alternatively the substrate contacting surface can be angled relative to the substrate facing surface.

The total contact surface area of the protrusions preferably does not exceed 20% of the total area of the substrate covered by the device. Even more preferably the contact surface area does not exceed 10% of the total area covered.

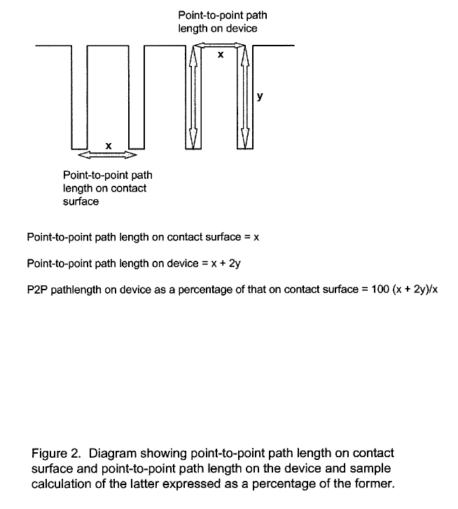

For applications to human or animal tissue, the point-to-point pathlength between adjacent contact points on the device should exceed the point-to-point pathlength on the device, as illustrated in FIG. 2.

In embodiments of the invention the point-to-point pathlength between adjacent contact points on the device exceeds the point-to-point pathlength on the device by 100%.

In embodiments of the invention the point-to-point pathlength between adjacent contact points on the device exceeds the point-to-point pathlength on the device by 200%.

In embodiments of the invention the point-to-point pathlength between adjacent contact points on the device exceeds the point-to-point pathlength on the device by 300%.

In embodiments of the invention the point-to-point pathlength between adjacent contact points on the device exceeds the point-to-point pathlength on the device by 400%.

For applications to human or animal tissue, the pathlength between adjacent contact points on the device should not exceed 1000% of the pathlength between the same points on the mobile surface (FIG. 2).

The attachment between the substrate contact surface of the protrusion and the flexible substrate may be generated by any means, including adhesive or mechanical means.

In embodiments of the invention the attachment is temporary.

The means of contact point attachment is preferably by adhesive or by the force generated by a local reduced pressure cavity. When an adhesive means of attachment is used, the adhesive is preferably a pressure sensitive adhesive, such as an acrylate-, polyurethane- or silicone-based adhesive.

In embodiments of the invention there is provided a medical device comprising or consisting of an attachment device according to the invention.

In embodiments of the invention there is provided a wound dressing according to the invention.

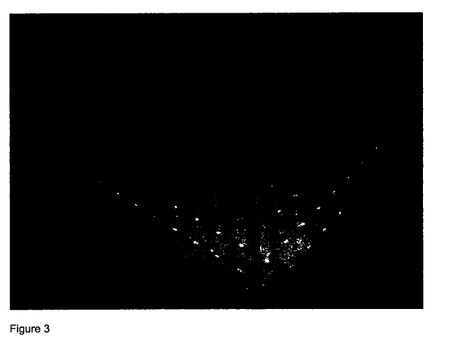

In an embodiment of the invention, the substrate facing surface forms the tissue contact layer of a topical wound management dressing. A pressure sensitive adhesive is coated onto at least the substrate contacting surface of the protrusion(s) (FIG. 3).

In another embodiment of the invention, the substrate facing surface forms the tissue contact layer of a transdermal delivery device, such as a patch. A pressure sensitive adhesive is coated onto substrate contacting surface.

In another embodiment of the invention, the substrate facing surface forms the tissue contact layer of a skin-facing electrode, such as those used in transcutaneous electrical nerve stimulation (TENS) devices or monitoring devices such as ECG and EEG readers. The contact layer is prepared from an electrically conductive polymer, such as a silicone-based elastomer of a formulation of suitable tack as would be applied by one skilled in the art for skin-facing electrodes (FIG. 3).

In another embodiment of the invention, the substrate facing surface forms the tissue contact layer of a contact lens. The protrusions are arranged in an array not obscuring the iris of the eye. This embodiment is advantageous because the low contact surface area does not hinder gaseous or liquid transport across the surface or the outer membrane of the eye.

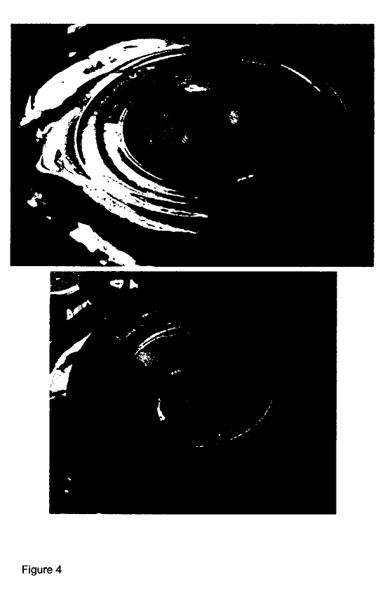

In another embodiment of the invention, the substrate facing surface forms the tissue contact layer of a covering enclosing a cavity for the application of reduced pressure. In this embodiment, a continuous projectile finned perimeter is preferable. More preferable is a concentric arrangement of projectile fins at the perimeter. The central area of the cover may also be finned but can also be point-projectile or blank. The central area of the cover may be of flat or curved profile, but is preferably of curved profile (e.g. hemispherical) to allow maximum spatial flexibility when extended by the adjoining tissue (FIG. 4).

In another embodiment of the invention, the projectile surface forms the tissue contact layer of a covering enclosing a cavity for the application of reduced pressure. In this embodiment, a continuous projectile finned surface is preferable. The projectile fins are positioned in a continuous geometrical pattern across the surface of the contact layer. Preferably, the geometry allows the device to be trimmed to shape while not forfeiting an air-tight perimeter tissue seal when placed under vacuum. Suitable geometries included concentric fins of circular or elliptical geometry.

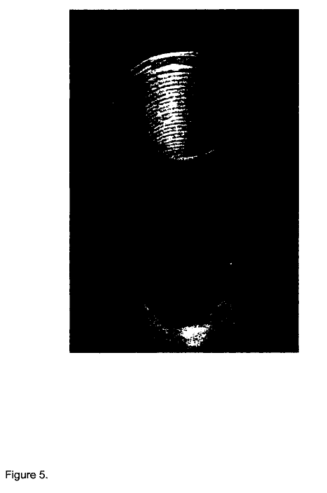

In another embodiment of the invention, the projectile surface forms the tissue contact layer of a covering enclosing a cavity for the application of reduced pressure. The device is of tubular geometry with concentric fins positioned on its inner surface (FIG. 5). The device is suitable elastic such that it can easily be located around limbs or bones and forms an effectively air-tight perimeter seal when placed under vacuum.

According to a second aspect of the invention there is provided a medical device consisting of or comprising of the attachment device according to the first aspect of the invention.

According to a third aspect of the invention there is provided a wound dressing consisting of or comprising of the attachment device according to the first aspect of the invention.

According to a fourth aspect of the invention there is provided an attachment device, medical device or wound dressing as substantially herein described with reference to the accompanying Examples and Figures.

The invention will now be described with reference to the following Figures, which are merely illustrative:

FIG. 1: Schematic illustrations of two embodiments of the invention. An example of point projection (left) and finned projections (right). A plan view diagrams and sample calculation of % surface area covered by contact points for the point projection are shown.

FIG. 2: Diagram showing point-to-point length on contact surface and point-to-point length on the device and sample calculation of the later expressed as a percentage of the former.

FIG. 3: Multiple point-projectile surface embodiment.

FIG. 4: Concentric-finned projection embodiment with hemispherical central section.

FIG. 5: Example of tubular device with concentric fin elements for circumferential attachment to limbs or bones

### DETAILED DESCRIPTION OF THE INVENTION

### Examples

### Example 1

### Device for the Application of Negative Pressure to a Tissue Surface

A heat-curable silicone elastomer was moulded in a concentric-finned geometry using a collapsible funnel (Normann, Copenhagen) in the compact position as a mould. A central aperture was made in the device and a luer fitting was inserted for tubing attachment. The resulting device is shown in FIG. 5.

### Example 2

### Comparison of the Device of Example 1 with a Traditional Suction Cup Geometry for the Application of Negative Pressure to an Intact Human Skin Surface

The device prepared in Example 1 was coupled to an intermittent (1 minute ON, 30 minutes OFF) vacuum source running at −200 mmHg relative to ambient atmospheric pressure. The vacuum source had a one-way valve, fitted to its exhaust to minimise vacuum loss during the OFF phase. During the ON phase, the device was placed gently against the skin of the abdomen. The device corrugated under the force of atmospheric pressure but the outermost sealing fin remained integral to the skin. The device, even in the absence of any adhesive, remained in place during repeated ON-OFF cycles. The device was left in place over 48 hours, including during normal tasks such as driving, removing and putting on clothing and sitting and standing.

For comparison, a soft silicone-based suction cup of traditional geometry (Cetacean Research Technology, Seattle, USA) was fitted with a luer fitting in a central position. This device was coupled to the vacuum source and put in tissue contact in an identical manner as above. The device remained in place during the initial ON phase but detached within seconds of the commencement of the first OFF phase.

The device of the invention remained in place for the period of the evaluation, in contrast to the traditional control, because it was able to maintain a sufficiently air-tight seal at its perimeter. It was able to achieve this because movements of the tissue surface (extensive, compressive and geometrical distortion) could be accommodated by flexing of the device in a manner that generated negligible force at the device perimeter seal. This is in contrast to a traditional cup geometry, where the perimeter seal can easily be mechanically displaced during movement of the tissue surface (particularly extension).

### Example 3

### Device for the Application of Negative Pressure to a Tissue Surface

A heat-curable silicone elastomer was moulded in a hemispherical geometry using-a double-walled mould. The set hemisphere was transferred to sit in the centre of a collapsible funnel (Normann, Copenhagen) in the compact position and additional heat-curable silicone elastomer was moulded around it. The resulting device had a central aperture made and a pressure-crackable valve (Minivalve International B.V.) was inserted into it. The resulting device is shown in FIG. 4.

### Example 4

### Comparison of the Device of Example 3 with a Traditional Suction Cup Geometry for the Application of Negative Pressure to an Intact Human Skin Surface

The device prepared in Example 3 was placed in contact with human abdominal tissue. The hemispherical dome section of the device was temporarily connected to a vacuum of −200 mmHg relative to ambient atmospheric pressure. When the dressing was fully compressed, the vacuum source was decoupled. The pressure-crackable valve maintained the level of vacuum initially obtained. The device remained in place for 3 hours before becoming detached when the contained vacuum decayed to a level unable to maintain the perimeter seal.

For comparison, a soft silicone-based suction cup of traditional geometry (Cetacean Research Technology, Seattle, USA) was fitted with a pressure-crackable valve in a central position. This device was coupled to the vacuum source and put in tissue contact in an identical manner as above. The device remained attached for under 30 seconds.

The device of the invention remained in place for 3 hours, in contrast to the traditional control, because it was able to maintain a sufficiently air-tight seal at its perimeter, as discussed in Example 2.

### Example 5

### Device for Attachment to Articulating Tissue

A heat-curable silicone elastomer was moulded in multiple point-projectile geometry. The points were positioned in a regularly repeating square unit-cell geometry. A silicone elastomeric pressure sensitive adhesive was coated into the tips of the projections. The resulting device is shown in FIG. 3.

1. An attachment device having a substrate facing surface, wherein the substrate facing surface is provided with at least one flexible protrusion extending therefrom, said protrusion forming a flexible bridge between the substrate facing surface and a flexible substrate.

2. An attachment device according to claim 1, wherein the device is provided with a first substrate facing surface and a second substrate facing surface, wherein both substrate facing surfaces are provided with at least one flexible protrusion extending therefrom, said protrusion forming a flexible bridge between the first substrate facing surface and a first flexible substrate and between the second substrate facing surface and a second flexible substrate.

3. An attachment device according to claim 1, wherein the substrate facing surface is provided with a plurality of protrusions.

4. An attachment device according to claim 3, wherein the protrusions extend substantially perpendicularly from the substrate facing surface.

5. An attachment device according to claim 3, wherein the protrusions are integral with the substrate facing surface.

6. An attachment device according to any of claim 3, wherein the protrusions are attached to the substrate facing surface.

7. An attachment device according to claim 3, wherein the geometry of the plurality of protrusions is identical.

8. An attachment device according to claim 1, wherein the point-to-point pathlength between adjacent contact points on the device should exceed the point-to-point pathlength on the device.

9. An attachment device according to claim 1, wherein the device is flexible.

10. An attachment device according to claim 1, wherein the substrate is soft tissue.

11. A medical device comprising an attachment device having a substrate facing surface, wherein the substrate facing surface is provided with at least one flexible protrusion extending therefrom, said protrusion forming a flexible bridge between the substrate facing surface and a flexible substrate.

12. (canceled)

13. A method of attaching a substrate to soft tissue comprising:
attaching a flexible protrusion of an attachment device to the soft tissue; and
coupling a substrate facing surface of the attachment device to the substrate.

14. The method of claim 13 wherein the substrate facing surface is coupled to the substrate via a second flexible protrusion.
